(19) United States
(12) Patent Application Publication
(10) Pub. No.: US 2010/0233795 A1
(43) Pub. Date: Sep. 16, 2010

(54) FERMENTATION VESSELS

(75) Inventor: Hans-Peter Mayer, Fulda (DE)

Correspondence Address: DAVIS & BUJOLD, P.L.L.C., 112 PLEASANT STREET, CONCORD, NH, 03301, US

(73) Assignee: JOHN CRANE UK LIMITED, Slough (GB)

(21) Appl. No.: 12/160,835

(22) PCT Filed: Dec. 27, 2006
(86) PCT No.: PCT/GB2006/004933

§ 371 Date: Sep. 30, 2008

(30) Foreign Application Priority Data
Jan. 14, 2006 (GB) 0600729.8

(51) Int. Cl. C12M 1/06; F16J 15/34

(52) U.S. Cl. 435/289.1; 277/377

(57) ABSTRACT

A fermentation apparatus (10) with a fermentation vessel (12) having a low speed stirrer comprising a shaft (14) with at least one stirring element (18) mounted thereon. First and second seal assemblies (30, 50), spaced axially from one another to define a chamber (64) therebetween, are provided to seal a gap between the shaft and a vessel aperture. An inlet (66) opens into the chamber for connecting the chamber to a processing gas, the first seal assembly (30) is located between the fermentation vessel and the chamber comprising a seat (32) mounted on the shaft for rotation therewith and a mating ring mounted (36) in fixed rotational relationship but moveably axially of the housing. A sealing face (44) of the mating ring is resiliently biassed into engagement with a sealing face (42) of the seat, and the sealing face has grooves (18) which provide separation of the sealing faces upon rotation of the shaft.

This is a national stage completion of PCT/GB2006/004933 filed Dec. 27, 2006 which claims priority from British Application Serial No. 0600729.8 filed Jan. 14, 2006.

### FIELD OF THE INVENTION

The present invention relates to fermentation vessels and in particular an apparatus for the aeration of submerged microbial or cell cultures, for biological purposes.

In such vessels a sufficient supply of oxygen is essential for the growth of aerobic micro-organisms as well as cell cultures and is therefore critical to obtain economic product yields in biotechnology using submerse fermentation processes. Standard oxygen supply of submerse fermentation processes is performed by introducing an oxygen containing gas via a stationary aeration ring at the bottom of a fermentation vessel. The efficiency of the fermentation process depends on the rate of transfer of oxygen across the gas/liquid interface to the liquid growth medium. This transfer rate depends mainly on the total interface surface, which depends on the size of the bubbles of oxygen introduced into the vessel, the smaller the bubbles, the higher the surface interface.

### BACKGROUND OF THE INVENTION

With conventional fermentation apparatus, the gas bubbles introduced into the bottom of the vessel, have a tendency to coagulate into larger bubbles, as the bubbles travel up the vessel. Anticoagulation chemicals may be used to reduce this effect. However such chemicals may adversely effect the product quality.

Hitherto an alternative solution to the problem of coagulation has been the use of mechanical stirrers to brake up larger bubbles. To be effective such stirrers require high speed mixing which may be detrimental to cell cultures used in modern biotechnology. Furthermore rapid stirring of the biological mass will induce heating, requiring cooling apparatus in order to maintain appropriate operating temperatures and therefore results in high energy costs.

European Patent Specification EP1479758 discloses a fermentation apparatus in which oxygen is introduced into the fermentation vessel via a hollow agitator shaft, at a plurality of distinct levels within the vessel, low speed stirring means being provided at each level. This construction enables an increase in the total gas/liquid interface to be achieved, at low stirring speeds. However this solution is relatively expensive and is only suitable for medium to large scale fermentation vessels.

### SUMMARY OF THE INVENTION

The present invention provides a cost effective solution to the problem of coagulation, which is suitable for small scale fermentation vessels.

Hereinafter the term processing gas shall mean a pure gas or a gas mixture that contains at least one component, said pure gas or at least one component being capable of being metabolised by micro-organisms in a fermentation process.

According to one aspect of the present invention, a fermentation apparatus comprises, a fermentation vessel having a low speed stirrer, the stirrer comprising a shaft entering the vessel through an aperture in the bottom of the vessel, one or more stirring elements being mounted on the shaft for rotation therewith, the or each stirring element being disposed within the vessel; and sealing means being provided to seal the gap between the shaft and aperture, said sealing means having a cylindrical housing attached to the bottom of the vessel, externally thereof and surrounding the shaft as it passes through the bottom of the vessel, first and second seal assemblies acting between the shaft and housing, are provided at axially spaced locations, to define a chamber therebetween, an inlet opening into said chamber for connection of the chamber to a supply of processing gas, the first seal assembly being located between the fermentation vessel and the chamber comprising a seat mounted on the shaft for rotation therewith and a mating ring mounted in fixed rotational relationship but moveably axially of the housing, a sealing face of the mating ring being resiliently biased into engagement with a sealing face of the seat, the sealing face of one of the mating ring or the seat having grooves, which will provide hydrodynamic separation of the sealing faces upon rotation of the shaft, allowing processing gas in the chamber to flow inwardly across the sealing faces and into the fermentation vessel, the grooves being bounded by a continuous dam formation adjacent the outer periphery of the mating ring or seat, so that when the shaft is stationary, the dam formation on the one sealing face will sealingly engage the other sealing face.

The faces of the seat and mating ring are configured to provide a hydrostatic and/or hydrodynamic separation between the sealing faces sufficient to allow an optimum passage of processing gas into the fermentation vessel to fulfil the requirements of the micro-organisms in use.

With the apparatus described above, when the shaft is rotating, hydrostatic/hydrodynamic effects will cause the seal faces to move apart, gas flowing between the seal faces from the chamber into the fermentation vessel. The processing gas which is fed to the chamber defined by the seal housing is thus fed into the fermentation vessel. Due to the narrow gap that is formed between the sealing faces and high shear forces, very small bubbles are formed in this manner and the centrifugal forces generated by rotation of the seat efficiently mix the bubbles with the liquid in the fermentation vessel. Both of these effects increase the gas/liquid interface in the fermentation vessel and therefore the transfer rate of the processing gas, even at low speeds. The seal faces may also be moved apart by hydrostatic forces, when pressure in the chamber is above a predetermined value, so that the processing gas may be fed to the fermentation vessel while there is sufficient pressure in the chamber, even though the shaft is not rotating. When the shaft stops rotating and the pressure of fluid in the chamber falls below the predetermined value, the resilient biasing of the seal rings will force the dam formation on one ring to sealingly engage the other ring to form a fluid tight seal.

The use of a hydrodynamic seal in accordance with the present invention also has the advantage that when the shaft is rotating the sealing faces are separated so that there will be no wear of the sealing faces, the debris of which may otherwise contaminate the product of the fermentation process.

According to a preferred embodiment of the invention, deflecting formations are provided on the external circumference of the seat adjacent the sealing face thereof, in order to enhance distribution of the bubbles in the fermentation vessel.

### BRIEF DESCRIPTION OF THE DRAWINGS

The invention is now described, by way of example only, with reference to the accompanying drawings, in which:—

FIG. 1 is a diagrammatic illustration in sectional elevation, of a fermentation apparatus in accordance with the present invention;

FIG. 2 shows a modification to the embodiment illustrated in FIG. 1; and

FIGS. 3 and 4 show further modifications to the apparatus shown in FIG. 1.

### DETAILED DESCRIPTION OF THE INVENTION

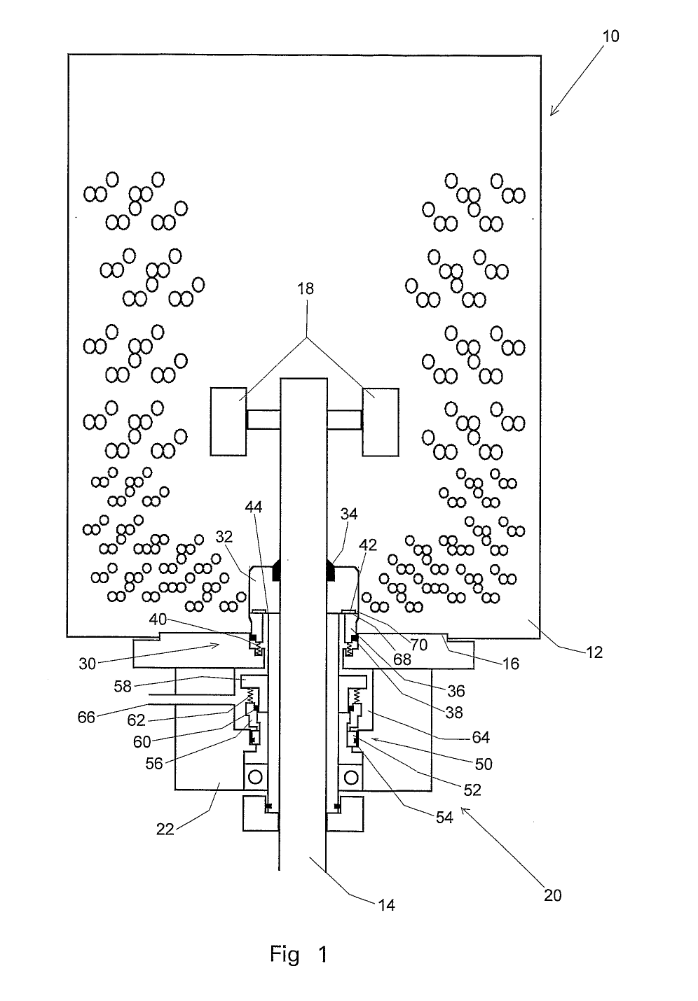

As shown in FIG. 1 a fermentation apparatus 10 comprises a fermentation vessel 12 in which, for example, a liquid microbial growth culture is contained. A shaft 14 is rotatably mounted through an aperture 16 in the bottom of the vessel 12. Mixing elements 18 are mounted on the shaft 14, within the vessel 12 and the shaft 14 is connected to drive means (not shown) externally of the vessel 12, by which the shaft 14 may be rotated at low speeds, to provide low energy stirring of the microbial growth culture.

The aperture 16 is closed by a hydrodynamic seal means 20, to provide a seal between the shaft 14 and vessel 12. The seal means 20 comprises a cylindrical housing 22 secured to the bottom of the vessel 12 and surrounding the shaft 14 as it enters the vessel 12. A first seal assembly 30 has a seat 32 which is mounted for rotation on shaft 14, within the fermentation vessel 12. The seat 32 is sealed with respect to the shaft 14 by elastomeric ring 34. A mating ring 36 is mounted on the housing 22 adjacent the inner end thereof. The mating ring 36 is secured to the housing to prevent rotation thereof, but is moveable axially of the housing 22. A sealing ring 38 slidably seals the mating ring 36 to the housing 22 and a series of angularly spaced helical compression springs 40 act axially between the housing 22 and mating ring 36, to urge a sealing face 42 of the mating ring 36, into sealing engagement with a sealing face 44 of the seat 32.

A second seal assembly 50 is mounted adjacent the outer end of housing 22. The second seal assembly 50 has a seat 52 mounted in fixed rotational and axial relationship to the housing 22 and sealed thereto by means of an elastomeric ring 54. A mating ring 56 is mounted on a carrier ring 58 on the shaft 14, the mating ring being rotationally fixed to the shaft but movably axially thereof, the mating ring being slidably sealed to the carrier ring 58 by sealing ring 60. A series of angularly spaced helical compression springs 62 act axially between the carrier ring 58 and mating ring 56 urging it into sealing engagement with the seat 52.

The first and second seal assemblies 30, 50 thereby define a chamber 64 externally of the fermentation vessel 12. An inlet 66 is provided to the chamber, the inlet 66 being connected to a source of processing gas.

The sealing face 44 of the seat 32 has a series of angularly spaced helical grooves 68. The grooves 68 extend outwardly, from a diameter radially inwardly of the internal diameter of the sealing face 42 of the mating ring 36 to a diameter radially inwardly of the external diameter of the sealing faces 42 and 44, the grooves 68 being inclined at an obtuse angle to the direction of rotation of the shaft 14. In this manner a continuous dam formation 70 is provided on the sealing face 44, which when the shaft 14 is stationary and pressure in the chamber 60 is below a predetermined value, is engaged by sealing face 42, to prevent leakage of the microbial growth culture, from the fermentation vessel 12.

When the shaft 14 rotates, processing gas in the chamber 64 is forced into the grooves 68, creating a high pressure zone adjacent the dam formation 70 and forcing the sealing faces 42, 44 apart to form a narrow gap between the sealing faces 42,44. The narrow gap permits the gas to flow between the sealing faces creating small bubbles in the microbial growth culture, the bubbles being distributed throughout the vessel 12 by the centrifugal action of the seat 32. Even when the shaft 14 is stationary, provided that the pressure of gas in chamber 60 is above a predetermined value, for example 2.5 bar, hydrostatic forces will open the seal faces 42,44 and allow gas to enter the vessel 12.

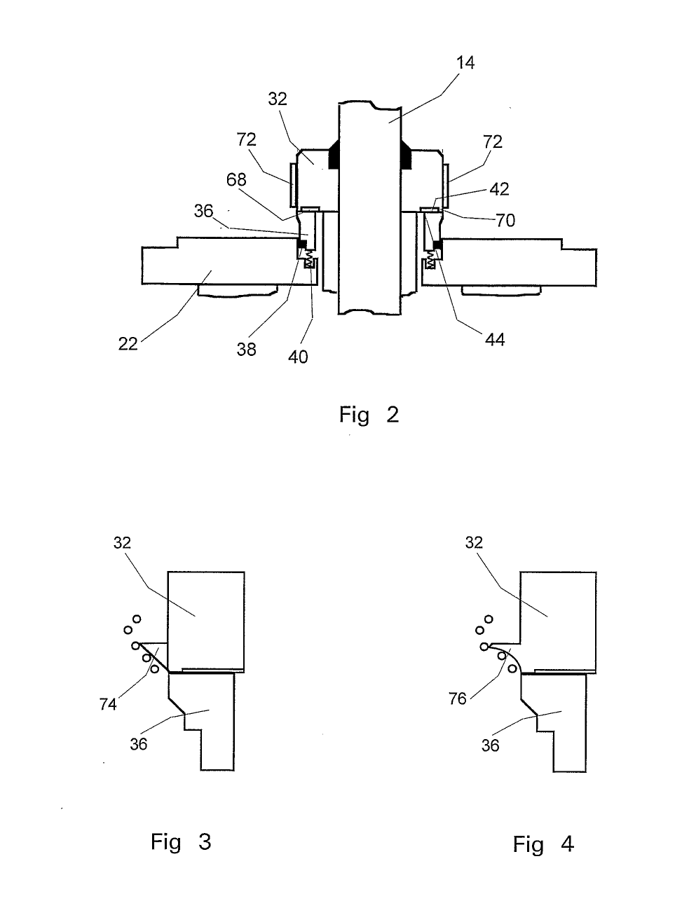

As illustrated on FIG. 2, axially extending vanes 72 may be provided of the circumferential surface of the seat 32 to augment or replace the mixing elements 18, to enhance the centrifugal effect and improve the distribution of the bubbles throughout the growth culture.

In the embodiments illustrated in FIGS. 3 and 4 wedge shaped annular formations 74, 76 are provided on the circumferential surface of the seat 32, adjacent the sealing face 44. The wedge formations 74, 76 will increase the diameter at which the bubbles detach from the seat 32 and thus the distribution of the bubbles throughout the growth culture. The wedge 74, 76 formations may be formed integrally of the seat 32 or may be formed separately of, for example, a low friction material, such as PTFE.

In order to inhibit gas bubbles from clinging to the sealing faces, the seat (32) and mating ring (36) are preferably made of low surface energy materials, or the sealing faces thereof may be coated with materials having low surface energy. Such materials include flurocarbons such as PTFE, or diamond and diamond like materials.

Various modifications may be included without departing from the invention. For example, while in the above embodiment, grooves 68 in the sealing face 44 provide hydrostatic as well as hydrodynamic separation, alternatively, the sealing faces 42,44 may be angled to provide hydrostatic separation of the faces 42,44, when pressure in the chamber 64 exceeds a predetermined value.

1-12. (canceled)

13. A fermentation apparatus (10) comprising:
a fermentation vessel (12) having a low speed stirrer, the stirrer comprising a shaft (14) mounted co-axially of the vessel (12) and entering the vessel (12) through an aperture (16) in a bottom of the vessel (12),
one or more stirring elements (18;72) being mounted on the shaft (14) for rotation therewith, the or each stirring element (18;72) being disposed within the vessel (12); and
sealing means (20) being provided to seal a gap between the shaft (14) and the aperture (16), the sealing means having a cylindrical housing (22) attached to the bottom of the vessel (12), externally thereof and surrounding the shaft (14) as it passes through the bottom of the vessel (12);
wherein first and second seal assemblies (30,50), acting between the shaft (14) and housing (22), are provided at axially spaced locations, to define a chamber (60) therebetween, an inlet (62) opening into the chamber (64) for connection of the chamber (60) to a supply of processing gas, the first seal assembly (30) is located between the fermentation vessel (12) and the chamber (64) comprising a first sealing ring (32) mounted on the shaft (14) for rotation therewith and a second sealing ring (36) mounted in fixed rotational relationship the housing (22), the first and second sealing rings are movable axially relative to one another and are resiliently biased towards one another, so that a sealing face (44) of the first sealing ring (36) may be biased into engagement with a sealing face (42) of the second sealing ring (32), the sealing face (42,44) of one of first and second sealing rings (32,34) has grooves (68), which will provide hydrodynamic separation of the sealing faces upon rotation of the shaft (14), allowing processing gas in the chamber (64) to flow inwardly across the sealing faces (42,44) and into the fermentation vessel (12), the grooves (68) are bounded by a continuous dam formation (70) adjacent the outer periphery of the sealing ring (32,34), so that when sealing face (42) engages sealing face (44) the dam formation (70) on the one sealing face (42,44) will sealingly engage the other sealing face (44,42).

14. The fermentation apparatus (10) in accordance with claim 13, wherein when the shaft is stationary and when pressure in the chamber is above a predetermined value, the sealing faces (42,44) are forced apart hydrostatically.

15. The fermentation apparatus (10) in accordance with claim 13, wherein the grooves (68) provide hydrostatic separation.

16. The fermentation apparatus (10) in accordance with claim 14, wherein the seal faces are angled to provide hydrostatic separation.

17. The fermentation apparatus in accordance with claim 13, wherein the sealing faces of one of a seat and a mating ring are formed from material having a low surface energy.

18. The fermentation apparatus (10) in accordance with claim 13, wherein one of a mating ring (36) and a seat (32) of the first seal assembly (30) has a series of angularly spaced helical grooves (68), the grooves (68) are inclined at an obtuse angle to the direction of rotation of the shaft (14), and inner ends of the grooves (68) are exposed to processing gas within the chamber (64).

19. The fermentation apparatus (10) in accordance with claim 13, wherein formations (72,74,76) are provided on a seat (32) of the first seal assembly (30) to enhance distribution of processing gas bubbles through a liquid within the vessel (12).

20. The fermentation apparatus (10) in accordance with claim 19, wherein angularly spaced, axially extending vanes (72) are provided on a circumferential surface of the seat (32) for one of augmenting and replacing the mixing elements (18).

21. The fermentation apparatus (10) in accordance with claim 19, wherein a wedge shaped annular formation (74,76) is provided on a circumferential surface of the seat (32), and the annular formation (74,76) is located adjacent the sealing face (44) of the seat (32).

22. The fermentation apparatus (10) in accordance with claim 21, wherein the annular formation (76) is formed integrally of the seat (32).

23. The fermentation apparatus (10) in accordance with claim 22, wherein the annular formation (74) is formed separately of the seat (32) and from a low friction material.

24. The fermentation apparatus (10) substantially as described herein, with reference to and as shown, in FIGS. 1 to 4.
